(19) United States
(12) Patent Application Publication
(10) Pub. No.: US 2010/0256757 A1
(43) Pub. Date: Oct. 7, 2010

(54) ARTIFICIAL SPHINCTER

(76) Inventors: Salvador Vilar Correia Lima, Recife (BR); Fabio De Oliveira Vilar, Recife (BR)

Correspondence Address: CANTOR COLBURN, LLP, 20 Church Street, 22nd Floor, Hartford, CT, 06103, US

(21) Appl. No.: 12/740,603

(22) PCT Filed: Aug. 29, 2008
(86) PCT No.: PCT/BR08/00321

§ 371 Date: May 26, 2010

(30) Foreign Application Priority Data
Oct. 31, 2007 (BR) PI0706143-9

(51) Int. Cl. A61F 2/08

(52) U.S. Cl. 623/14.13

(57) ABSTRACT

Artificial sphincter, the present invention refers to an artificial sphincter for treating urinary incontinence, and more specifically, an artificial sphincter to be implanted in a live being, particularly a male human being for treating urinary incontinence. According to one aspect of the present invention, the artificial sphincter (10) comprises a cuff (M) intended to surround at least part of the urethra of an individual, said cuff being in fluid communication with an unidirectional displacement pump (B1), said artificial sphincter (1) comprising additionally a second pump (B2) in fluid communication with said first pump and said cuff, and a third pump (B3) in fluid communication with said cuff.

### FIELD OF THE INVENTION

The present invention refers in general to an artificial sphincter for treating urinary incontinence. More specifically, the present invention refers to an artificial sphincter to be implanted in a live being, particularly a male human being for treating urinary incontinence.

### BACKGROUND OF THE INVENTION

The act of urinating is a complex function of the human being body, which is carried out as a result of the inter-relationship of parts of the nervous system and muscles, thus forming a system still not totally understood by the science.

The loss of the capacity of urination control, whether caused by involuntary leakage of urine or interruption, at different degrees, is called urinary incontinence. The publication Fourth Report on the Standardization of Terminology of Lower Urinary Tract Function, International Continence Society Standardization Committee, 1949, defines the urinary incontinence as an involuntary loss of urine which may be objectively shown causing a hygienic and/or social problem.

Briefly, the urinary system may be outlined as being formed by smooth, striated muscle fibers which contract and relax in order to keep a pressure gradient which secures the urine will be held in bladder, and its release at the convenient time. Despite there are terminations of sympathetic and parasympathetic systems distributed all over the bladder and sphincteral region, it's known that the vesical muscle (detrusor) is rich of cholinergic receptors, and the sphincteral region is mainly controlled by alpha-1 adrenergic receptors. The integration with the voluntary nervous system is made by means of the pudendal nerve, whose major activity would at first be the voluntary interruption of urination.

A failure of such mechanism, which is called sphincter, causes most varied degrees of urinary incontinence. Changes in the sphincter functioning, particularly in male individuals, may be caused by neurological disorders, such as Parkinson's disease, rachimedullar trauma and stroke; congenital defects, or even chronic diseases like diabetes.

Mild changes in the complex working of the sphincter may be treated with medicines, which inhibit or stimulate the terminations of the sympathetic and parasympathetic systems. Intermediate processes, when there are primarily anatomic dysfunctions, like those of the straphia-epispadia complex, include surgical procedures whose purpose is to reinforce these mechanisms by elongating or slanting the posterior urethra and vesical colon.

The use of urethral “Slings” for supporting the urethra also shows to be effective in treating urinary incontinence, but such procedure involves a high rate of urethra erosion.

The injection of gelatinous substances around the urethra is another method used, but the high rates of infection, rejection and development of safer methods have decreased the application of gelatinous substances.

Prostate diseases and their treatments are the most frequent causes of urinary incontinence in individuals, particularly male individuals, especially in cases of prostate cancer. The prostate removal procedure (radical prostatectomy) is the major cause of incontinence. It's believed that urinary incontinence occurs in 2 to 60 percent of male individuals who had a radical prostatectomy surgery.

Although the state of the art shows some treatments that try to resolve urinary incontinence, as described above, many times there are situations in which the problem persists, and so the individual, particularly a male individual, will have failures in the sphincteral mechanism, thus resulting in the persistence of the urinary incontinence.

In these cases, the alternative for reinforcing the mechanisms of urination control is the use of an artificial sphincter, also called mechanical sphincter.

The state of the art discloses a number of artificial sphincters designed to resolve urinary incontinence, particularly for male individuals.

Scott et al., 1973, describe an artificial sphincter comprising essentially a pressure regulating reservoir, which is placed in the abdominal cavity of the individual, connected to a pump which transfers liquid from inside the reservoir to a cuff placed around the urethra. The pressure generated by the balloon is exerted over the cuff which then holds the urethra occluded during urination intervals. When the patient has to urinate, he/she squeezes the pump with his/her fingers to transfer the liquid from the cuff to the abdominal reservoir, which in its turn returns it slowly to the cuff, thus resuming occlusion of the urethra, and keeping the same pressure gradient. The problem with this kind of artificial sphincter is the frequent necrosis of the urethra regions due to the great pressure exerted by the cuff.

The sphincter model AS-800, marketed by American Medical System, Minnesota, USA, solves in part the problem of necrosis of the urethra regions. However, this device is relatively expensive because a large number of components are used. Besides, this type of solution is not effective in case of an eventual abdominal effort, when, for example, the individual coughs or bends his/her body forwards, contracting the abdomen. In such situations, the additional effort placed over the abdomen may generate a pressure over the bladder, and the pressure exerted by the cuff over the urethra may not be enough to hold the urine, thus generating an involuntary leakage of urine.

Therefore, the objective of the present invention is to overcome the problems shown by other treatment methods and artificial sphincters belonging to the state of the art.

More particularly, the object of the present invention is an artificial sphincter capable of solving problems shown by other sphincters of the art, particularly for the treatment of urinary incontinence of a male individual.

More particularly, the object of the present invention is an artificial sphincter which is relatively inexpensive when compared to the sphincters of the art, because it comprises a smaller number of components or less complex components, thus decreasing costs and making easier its manufacture.

Still another object of the present invention is an artificial sphincter which prevents, or at least avoids, involuntary leakage of urine when the individual eventually makes an abdominal effort.

### DESCRIPTION OF THE INVENTION

The above-mentioned objectives, among others, are achieved with the present invention.

According to a first aspect, the present invention refers to an artificial sphincter comprising a cuff, intended to surround at least part of the urethra of an individual, said cuff being in fluid communication with a unidirectional displacement pump, characterized in that said sphincter comprises additionally a second pump in fluid communication with said first pump and said cuff, and a third pump in fluid communication with said cuff.

The present invention may feature one or more of the following aspects:
A reservoir which is in fluid communication with said third pump;
Said second pump is a unidirectional displacement pump;
Said second pump comprises a restrictor for slow release of the fluid back to the cuff;
The slow release represents a time delay comprised between 10 s and 3 min, particularly between 20 s and 2 min, more particularly between 30 s and 1 min, and even more particularly around 45 s;
Said cuff is implanted in the posterior urethra next to the vesical colon;
Said cuff is implanted at the level of the bulbar urethra;
Said first pump is implanted in the scrotum of a male individual;
Said second pump is implanted in the inguinal channel of a male individual;
Said third pump is implanted in the abdominal cavity of a male individual;
Said reservoir is implanted in the subcutaneous space between the anterosuperior iliac crest and the navel of a male individual.
The fluid communication between said pumps, the cuff and eventually the reservoir is made by means of conduits;
Said fluid is distilled water;
The system pressure when filled with fluid is comprised between approximately 20 and 150 cmH₂O, more particularly between approximately 40 and 100 cmH₂O, even more particularly between 50 and 90 cmH₂O, more particularly about 70 cmH₂O .
The sphincter of the invention is made of a medical grade silicone.
The sphincter of the present invention may also be implanted in female individuals and children, after making common adaptations within the reach of any person skilled in the art.

### BRIEF DESCRIPTION OF THE DRAWING

These and other objects, improvements and effects of the sphincter of the present invention will become apparent to those skilled in the art from the detailed description shown below, with reference to the attached drawings given with an illustration purpose only of a particular embodiment of the invention. Said drawings are schematic views and their dimensions or proportions may not correspond to reality, once they are intended to illustrate the invention didactically only, without imposing any limitations but those of the claims included herein below, being that:

FIG. 1 is a schematic representation of the artificial sphincter according to an embodiment of the present invention; and

FIGS. 2 and 3 are details of the cuff comprised by the sphincter of FIG. 1 showing their closed and open positions, respectively.

### DESCRIPTION OF PARTICULAR EMBODIMENTS

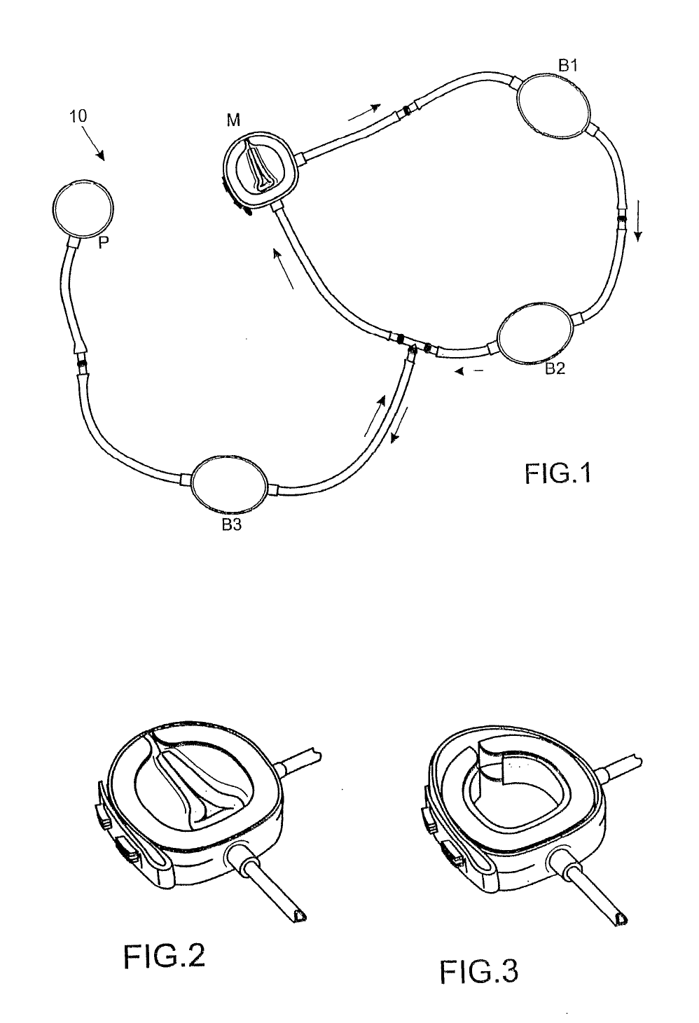

FIG. 1 shows schematically an artificial sphincter according to an embodiment of the present invention. The sphincter of the invention comprises a cuff (M) intended to surround at least part of the urethra of an individual. According to the embodiment represented herein, and according to FIGS. 2 and 3, the cuff (M) is intended to surround the totality of the urethra of an individual.

Said cuff (M) comprises opening and closing means for opening the cuff, surrounding the urethra and closing the cuff kept around the urethra of an individual. Said cuff comprises a bag which may be filled or emptied with a fluid. When filled with a fluid at a given pressure, the cuff stays in an occluded position, as shown in FIG. 2, and when emptied, the cuff opens, decreasing the pressure around the urethra, and thus allowing the urine to pass through it.

The cuff must be implanted around the posterior urethra next to the vesical colon, or at the level of the bulbar urethra.

The cuff may be made of a flexible, medical grade silicone material whose inner face is coated with a polyurethane foam strip, and the outer face is reinforced with a polyester strap. Typically, the cuff is adjustable, and those commonly found in the market have three different sizes: 55, 65 and 75 mm of inner perimeter.

More details of the cuff are not necessary in the present description, as this device is largely known by those skilled in the art.

The cuff is in a fluid communication with a first pump (B1). Said first pump is preferably a unidirectional displacement pump, i.e., when actuated, the fluid contained in the sphincter may only be displaced in one direction (represented by the arrows of FIG. 1), i.e. towards the second pump (B2), which will be described in more details below.

The purpose of said first pump is to empty the cuff, what means, releasing the fluid contained therein, and consequently, the pressure exerted by the fluid over its walls, thus allowing the cuff to be open and the urine coming from the bladder to pass through the urethra.

The pump (B1) is preferably implanted in the scrotum of a male individual, and its actuation is made by squeezing the walls with the fingers.

More detailed working of the sphincter of the invention will be described later.

A second pump (B2) is in fluid communication with the first pump (B1) and the cuff (M). The second pump receives the fluid coming from the cuff (M) and the first pump (B1), when the first pump is actuated, and the purpose is to return the fluid to the cuff to fill it again, but slowly. In other words, the fluid coming from the cuff, passes by the first pump (B1), when it is actuated, and houses in the second pump (B2), but it is not immediately returned to the cuff. Therefore, the fluid is slowly returned to the cuff. Such slower return may be provided for example by means of the outlet decreased diameter of the second pump (B2), thus decreasing the fluid flow, and consequently, allowing slow filling of the cuff.

This characteristic is advantageous so that the individual having an artificial sphincter according to the present description will be able to actuate the first pump (B1) only once to empty the cuff and allow urination. As the cuff is refilled slowly, the individual may urinate again without needing to actuate the first pump (B1) again up to emptying the bladder.

In the present specification, the word “slow” or “slow release” means a time delay comprised between 10 s and 3 min, particularly between 20 s and 2 min, more particularly between 30 s and 1 min, and even more particularly about 45 s.

The second pump (B2) is preferably implanted in the inguinal channel of a male individual.

A third pump (B3) is in fluid communication with the cuff (M). This third pump (B3) is preferably implanted in the abdominal cavity of an individual. The objective of using this third pump is to increase the cuff pressure when it is squeezed, particularly when the individual eventually makes an effort over his/her abdomen, as it is the case, for example, when coughing or bending his/her body forwards, thus contracting the abdomen. The additional pressure exerted by the third pump (B3) over the cuff only occurs when squeezed, what means, in case of an eventual abdominal effort, thus avoiding such higher abdominal pressure to be exerted also over the bladder, and causing an involuntary passage of urine through the cuff occluding the urethra. Since such higher pressure exerted by the pump (B3) over the cuff is only eventual, i.e. when there is an increased abdominal effort, the problem of necrosis of the urethra does not exist.

The third pump is in fluid communication with a reservoir (P), whose objective is to activate and/or deactivate the system. Such reservoir (P) is preferably implanted in the subcutaneous space between the anterosuperior iliac crest and the navel, particularly in a male individual.

The working of the sphincter of the present invention is now explained in more details.

While in rest, the whole system is kept full of fluid at a preset pressure, which allows the continence. When squeezed, the first pump (B1) allows emptying the cuff, and consequently, it's open by the displacement of the fluid contained therein towards it. Upon emptying and opening the cuff, urination occurs.

The fluid contained in the first pump (B1) is transferred to the second pump (B2) where it is kept while it is slowly released again to the cuff, which is little by little filled again with fluid and occluded, squeezing the urethra, and securing continence again.

As mentioned above, the function of the third pump (B3) is to increase the system pressure, and consequently the cuff pressure, when squeezed by an occasional abdominal effort.

The objective of reservoir (P) is to activate and deactivate the system as mentioned above. When the surgery for implanting an artificial sphincter is done, the sphincter is not filled with fluid immediately. The body needs to regenerate before the system is activated. Therefore, the system is only filled with fluid, and consequently, its activation will only occur typically 6 to 8 weeks after the implantation surgery.

In such a regard, it is required a convenient means for having access to the artificial sphincter to fill it with fluid and provide the system with a suitable pressure. With the reservoir implanted in the subcutaneous space between the anterosuperior iliac crest and the navel, the doctor who will fill it with fluid will have easy access to the system, without the need of a new surgical intervention to fill the sphincter with fluid. Particularly, the equipment used for saline infusion may be employed for filling the sphincter of the present invention with fluid. The fluid is preferably distilled water.

It must be provided fluid enough in order to exert a pressure over the system between approximately 20 and 150 cmH₂O, more particularly between approximately 40 and 100 cmH₂O, even more particularly between 50 and 90 cmH₂O, more particularly about 70 cmH₂O.

The fluid communication between the pumps (B1-B3), the cuff (M) and the reservoir (P) may be made by means of conduits allowing the fluid to pass.

Both pumps (B1-B3) and the reservoir (P) and the conduits are made of a medical grade silicone.

While the above description of the particular embodiment is directed to the implantation of a sphincter in a male individual, it should be made clear that the sphincter of the present invention can also be implanted in female individuals and children, provided the required adaptations within the reach of any person skilled in the art are made.

It should be recognized that even though the present invention has been described in connection with a particular embodiment, people skilled in the art may develop a large variation of details and expand the aforementioned sphincter to other types of applications without, however, departing from the principles of the invention. For example, various elements shown in the embodiment may be arranged in a manner not illustrated above and according to the attached drawings. Therefore, the claims included below should be construed as covering all the equivalents falling within the scope and character of the invention.

1. “ARTIFICIAL SPHINCTER” (10) which comprises a cuff (M) intended to surround at least part of the urethra of an individual, said cuff being in fluid communication with a unidirectional displacement pump (B1), characterized in that said sphincter comprises additionally a second pump (B2) in fluid communication with said first pump and said cuff, and a third pump (B3) in fluid communication with the cuff.

2. “ARTIFICIAL SPHINCTER” (10), according to claim 1, characterized in that it comprises additionally a reservoir (P) in fluid communication with said third pump (B3).

3. “ARTIFICIAL SPHINCTER” (10), according to claim 1, characterized in that said second pump (B2) is a unidirectional displacement pump.

4. “ARTIFICIAL SPHINCTER” (10), according to claim 1, characterized in that said second pump (B2) comprises a restrictor for slow release of the fluid around the cuff (M).

5. “ARTIFICIAL SPHINCTER” (10), according to claim 4, characterized in that said slow release represents a time delay comprised between 10 s and 3 min, particularly 20 s and 2 min, more particularly between 30 s and 1 min, and even more particularly about 45 s.

6. “ARTIFICIAL SPHINCTER” (10), according to claim 1, characterized in that said cuff is implanted in the posterior urethra next to the vesical colon of a male individual.

7. “ARTIFICIAL SPHINCTER” (10), according to claim 1, characterized in that said cuff is implanted at the level of the bulbar urethra of a male individual.

8. “ARTIFICIAL SPHINCTER” (10), according to claim 1, characterized in that said first pump (B1) is implanted in the scrotum of a male individual.

9. “ARTIFICIAL SPHINCTER” (10), according to claim 1, characterized in that said second pump (B2) is implanted in the inguinal channel of a male individual.

10. “ARTIFICIAL SPHINCTER” (10), according to claim 1, characterized in that said third pump (B3) is implanted in the abdominal cavity of a male individual.

11. “ARTIFICIAL SPHINCTER” (10), according to claim 2, characterized in that said reservoir (M) is implanted in the subcutaneous space between the antero superior iliac crest and the navel of a male individual.

12. “ARTIFICIAL SPHINCTER” (10), according to claim 1, characterized in that the fluid communication between said pumps (B1-B3), the cuff (M), and eventually the reservoir (P) is made by conduits.

13. “ARTIFICIAL SPHINCTER” (10), according to claim 1, characterized in that the fluid is distilled water.

14. “ARTIFICIAL SPHINCTER” (10), according to claim 1, characterized in that the system pressure is comprised between approximately 20 and 150 cmH₂O, more particularly between approximately 40 and 100 cmH₂O, even more particularly between 50 and 90 cmH₂O, more particularly about 70 cmH₂O.

15. “ARTIFICIAL SPHINCTER” (10), according to claim 1, characterized in that it is made of a medical grade silicone.

16. “ARTIFICIAL SPHINCTER” (10), according to claim 1, characterized in that it is implanted in a male individual.

17. “ARTIFICIAL SPHINCTER” (10), according to claim 1, characterized in that it is implanted in a female individual.
